(19) United States
(12) Patent Application Publication
(10) Pub. No.: US 2026/0345357 A1
(43) Pub. Date: Nov. 5, 2026

(54) ENDOPROSTHESIS AND METHOD OF MAKING AN ENDOPROSTHESIS

(71) Applicant: Boston Scientific Scimed, Inc., Maple Grove, MN (US)

(72) Inventors: Javier Palomar-Moreno, Galway (IE); Anthony Malone, Galway (IE)

(73) Assignee: Boston Scientific Scimed, Inc., Maple Grove, MN (US)

(21) Appl. No.: 19/666,849

(22) Filed: May 4, 2026

Related U.S. Application Data
(60) Provisional application No. 63/799,950, filed on May 5, 2025.

(51) Int. Cl. A61F 2/915

(52) U.S. Cl. CPC A61F 2/915; A61F 2002/91575; A61F 2220/0025; A61F 2230/0006; A61F 2230/0091; A61F 2240/001; A61F 2250/0029; A61F 2250/0036

(57) ABSTRACT

An endoprosthesis may include a plurality of circumferential bands, each including a plurality of struts joined in a serpentine pattern, and a plurality of connecting members extending between adjacent bands. Each strut of a subset of the plurality of struts includes a gap formed at a location having low stress during expansion of the endoprosthesis. Locations adjacent the location having low stress experience high stress during expansion. The gap may be formed along a straight portion of the strut, and separate the strut into first and second portions. The gap is filled with a biodegradable polymeric material coupling the first and second portions. A method of making the endoprosthesis includes forming the bands and the connecting members from a metallic tubular member, cutting a first strut to form a first gap, filling the first gap, cutting a second strut to form a second gap, and filling the second gap.

### CROSS-REFERENCE TO RELATED APPLICATION

This application claims the benefit of U.S. Patent Application Serial No. 63/799,950, filed May 5, 2025, entitled "ENDOPROSTHESIS AND METHOD OF MAKING AN ENDOPROSTHESIS”, which is incorporated by reference herein in its entirety.

### TECHNICAL FIELD

The present disclosure pertains to medical devices, methods for manufacturing medical devices, and uses thereof. More particularly, the present disclosure pertains to a stent or endoprosthesis for implantation in a body lumen, and associated methods.

### BACKGROUND

A stent or endoprosthesis may be used in the treatment of body lumens. A stent is a generally longitudinal tubular device formed of biocompatible material which is useful to open and support various lumens in the body. For example, stents may be used in the vascular system, urogenital tract, gastrointestinal tract, esophageal tract, renal tract, tracheal/bronchial tubes, and bile duct, as well as in a variety of other applications in the body.

Placement of a stent or endoprosthesis within a blood vessel or a body lumen has been shown to effectively open the blood vessel or body lumen. However, due to the structure and/or radial strength of the stent or endoprosthesis, normal motion and/or dynamic function of the blood vessel or body lumen (e.g., vasomotion, etc.) may be suppressed. In some situations, it may be desirable to restore normal motion and/or dynamic function to the blood vessel or body lumen after a suitable amount of time (e.g., weeks/months post-implantation, etc.). Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, delivery method, and use alternatives for medical devices such stents or endoprostheses.

In one example, an endoprosthesis configured to shift between a radially collapsed configuration and a radially expanded configuration may comprise a plurality of circumferential bands, wherein each circumferential band comprises a plurality of struts joined in a serpentine pattern to define peaks and valleys, and a plurality of connecting members extending between adjacent circumferential bands of the plurality of circumferential bands. Each strut of a subset of the plurality of struts may comprise a gap formed at a location having low stress during expansion of the endoprosthesis. Locations adjacent the location having low stress may experience high stress during expansion of the endoprosthesis.

In addition, or alternatively, to any example disclosed herein, the gap extends across an entire width of its respective strut.

In addition, or alternatively, to any example disclosed herein, each strut is defined by a peak at one end and a valley at an opposing end.

In addition, or alternatively, to any example disclosed herein, the location having low stress during expansion of the endoprosthesis is disposed between the peak and the valley.

In addition, or alternatively, to any example disclosed herein, the location having low stress during expansion of the endoprosthesis is disposed equidistantly between the peak and the valley.

In addition, or alternatively, to any example disclosed herein, the locations adjacent the location having low stress during expansion of the endoprosthesis are disposed between the peak and the valley.

In addition, or alternatively, to any example disclosed herein, the locations adjacent the location having low stress during expansion of the endoprosthesis are disposed immediately adjacent the peak and the valley.

In addition, or alternatively, to any example disclosed herein, the plurality of struts has a first width, and the peaks and valleys have a second width greater than the first width.

In addition, or alternatively, to any example disclosed herein, the gap is filled with a biodegradable polymeric material.

In addition, or alternatively, to any example disclosed herein, the plurality of circumferential bands and the plurality of connecting members are cut from a metallic tubular member.

In addition, or alternatively, to any example disclosed herein, the subset of the plurality of struts is arranged helically around a central longitudinal axis of the endoprosthesis.

In addition, or alternatively, to any example disclosed herein, the subset of the plurality of struts divides the endoprosthesis into at least two distinct helical stent structures.

In addition, or alternatively, to any example disclosed herein, and in a second example, a method of making an endoprosthesis configured to shift between a radially collapsed configuration and a radially expanded configuration may comprise: forming a plurality of circumferential bands, wherein each circumferential band comprises a plurality of struts joined in a serpentine pattern to define peaks and valleys, and a plurality of connecting members extending between adjacent circumferential bands of the plurality of circumferential bands from a metallic tubular member to form the endoprosthesis; cutting a first strut of the plurality of struts to form a first gap at a location along the first strut having low stress during expansion of the endoprosthesis; filling the first gap with a biodegradable polymeric material; after filling the first gap, cutting a second strut of the plurality of struts to form a second gap at a location along the second strut having low stress during expansion of the endoprosthesis; and filling the second gap with the biodegradable polymeric material.

In addition, or alternatively, to any example disclosed herein, the method may comprise: cutting a subset of struts of the plurality of struts to form gaps at locations having low stress during expansion of the endoprosthesis, wherein the subset of struts is arranged helically around a central longitudinal axis of the endoprosthesis; and filling the gaps with the biodegradable polymeric material. Each gap may be cut and then filled prior to cutting a subsequent gap.

In addition, or alternatively, to any example disclosed herein, the endoprosthesis defines at least three distinct helical stent structures coupled together by the biodegradable polymer to form a substantially cylindrical structure.

In addition, or alternatively, to any example disclosed herein, the biodegradable polymeric material is configured to degrade after a predetermined period of time, thereby permitting the at least three distinct helical stent structures to separate from each other.

In addition, or alternatively, to any example disclosed herein, the cutting and filling steps occur while the endoprosthesis is disposed in the radially collapsed configuration.

In addition, or alternatively, to any example disclosed herein, the plurality of circumferential bands and the plurality of connecting members are monolithically formed with each other prior to cutting any struts.

In addition, or alternatively, to any example disclosed herein, and in a third example, an endoprosthesis configured to shift between a radially collapsed configuration and a radially expanded configuration may comprise a plurality of circumferential bands, wherein each circumferential band comprises a plurality of struts joined in a serpentine pattern to define peaks and valleys, and a plurality of connecting members extending between adjacent circumferential bands of the plurality of circumferential bands. Each strut of a subset of the plurality of struts may comprise a gap formed along a straight portion of the strut, the gap separating the strut into a first portion and a second portion. The gap may be filled with a biodegradable polymeric material coupling the first portion to the second portion.

In addition, or alternatively, to any example disclosed herein, the plurality of circumferential bands and the plurality of connecting members are formed from a metallic tubular member.

The above summary of some embodiments, aspects, and/or examples is not intended to describe each disclosed embodiment or every implementation of the present disclosure. The figures and detailed description which follow more particularly exemplify these embodiments .

### BRIEF DESCRIPTION OF THE DRAWINGS

The disclosure may be more completely understood in consideration of the following detailed description in connection with the accompanying drawings, in which:

FIG. 1 is a flat pattern view of an endoprosthesis;

FIGS. 2-3 are side views schematically illustrating expansion of the endoprosthesis of FIG. 1 from a radially collapsed configuration to a radially expanded configuration;

FIG. 4 is detail view illustrating selected aspects of a portion of the endoprosthesis of FIG. 3;

FIG. 5 is a flat pattern view selected aspects of the endoprosthesis of FIG. 1 in accordance with the disclosure;

FIGS. 6-10 illustrate selected aspects of alternative configurations of the endoprosthesis of FIG. 5; and

FIGS. 11-16 schematically illustrate selected aspects of a method of making an endoprosthesis according to the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

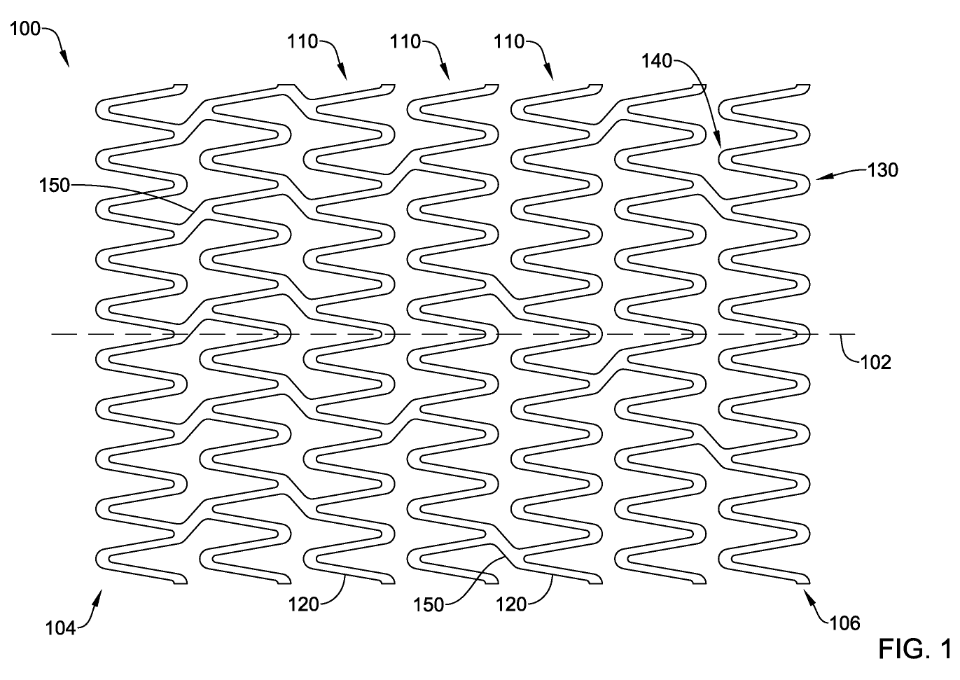

FIG. 1 is flat pattern view illustrating selected aspects and/or details of an endoprosthesis 100 (e.g., a stent). The disclosure describes aspects of the endoprosthesis 100, otherwise referred to as a stent, and a method or methods of making the endoprosthesis 100. In one example, the endoprosthesis 100 may be used to treat a stenosis in a blood vessel, may be used to maintain a fluid opening or pathway in the vascular, urinary, biliary, tracheobronchial, esophageal, gastrointestinal, or renal tracts, or in some alternative configurations may be used to position a device such as an artificial valve or a filter within a body lumen. In some instances, the endoprosthesis 100 may be a prosthetic graft, a stent-graft, or a stent (e.g., a vascular stent, tracheal stent, bronchial stent, esophageal stent, gastrointestinal stent, biliary stent, etc.). For the purposes of this disclosure, the terms “endoprosthesis” and “stent” may generally be used interchangeably. The endoprosthesis 100 may be introduced endoscopically, subcutaneously, percutaneously, or surgically to be positioned within an organ, tissue, or lumen, such as a heart, artery, vein, urethra, esophagus, trachea, bronchus, bile duct, or the like.

For the purpose of illustration only, the endoprosthesis 100 is described in the context of treating and/or supporting a body lumen, such as an artery, that normally has biological dynamism (e.g., vasomotion, hemodynamic modulation, etc.). However, the skilled artisan will recognize that the endoprosthesis 100 may have other uses and/or may be used to treat other body lumens and/or locations with little or no change to the disclosed structure(s).

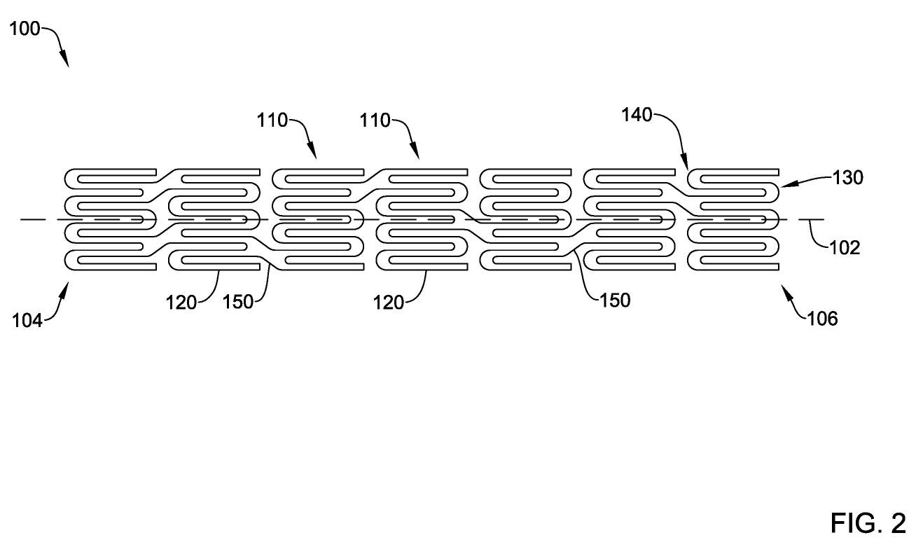

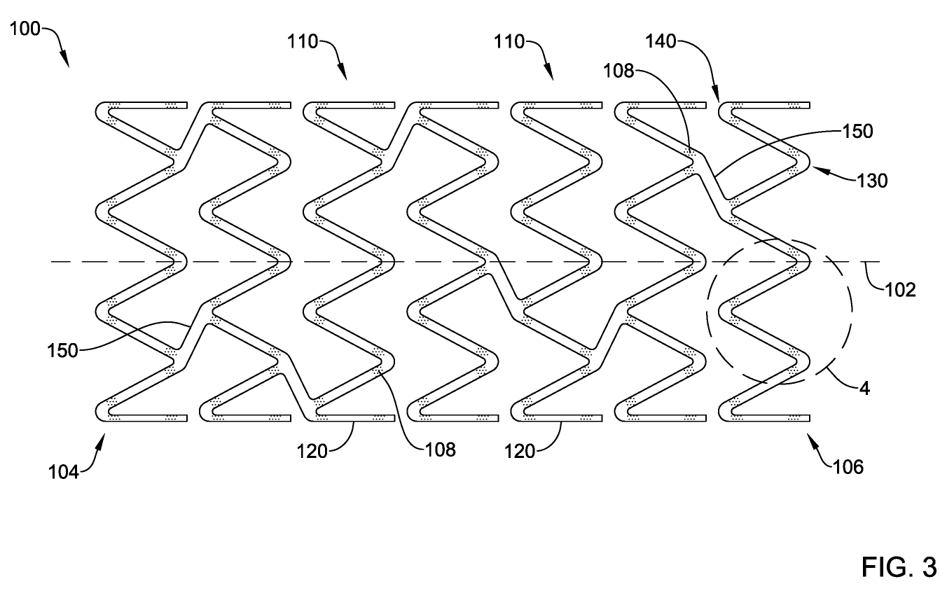

The endoprosthesis 100 may be configured to shift between a radially collapsed configuration (e.g., FIG. 2) and a radially expanded configuration (e.g., FIG. 3). The flat pattern view shown in FIG. 1 illustrates the endoprosthesis 100 in the radially expanded configuration. In some embodiments, the endoprosthesis 100 may define a lumen extending therethrough along a central longitudinal axis 102 from a proximal end 104 to a distal end 106. In some embodiments, the endoprosthesis 100 may have and/or may define a substantially cylindrical outer surface and/or a substantially cylindrical structure. In some embodiments, the endoprosthesis 100 may define at least two distinct helical stent structures coupled together to form the substantially cylindrical structure, as discussed in more detail below. In some embodiments, the endoprosthesis 100 may define at least three distinct helical stent structures coupled together to form the substantially cylindrical structure. Other configurations are also contemplated.

In some embodiments, the endoprosthesis 100 may comprise a plurality of circumferential bands 110, wherein each circumferential band of the plurality of circumferential bands 110 comprises a plurality of struts 120 joined in a serpentine pattern, a sinusoidal pattern, an undulating pattern, and/or a zig-zag pattern to form and/or define peaks 130 and valleys 140 at opposing ends of each strut of the plurality of struts 120. The peaks 130 and the valleys 140 may be offset from each other in a longitudinal and/or axial direction. Adjacent struts of the plurality of struts 120 within each circumferential band may be fixedly attached to each other at the peaks 130 and/or the valleys 140 of their respective circumferential band. In some embodiments, the plurality of struts 120 within each circumferential band may be oriented generally parallel to each other and/or the central longitudinal axis 102 of the endoprosthesis 100 in the radially collapsed configuration. In some embodiments, the plurality of struts 120 within each circumferential band may be oriented non-parallel to each other and/or the central longitudinal axis 102 of the endoprosthesis 100 in the radially expanded configuration. Other configurations are also contemplated.

In some embodiments, the endoprosthesis 100 may comprise a plurality of connecting members 150 extending between adjacent circumferential bands of the plurality of circumferential bands 110. The plurality of connecting members 150 may fixedly attach adjacent circumferential bands of the plurality of circumferential bands 110 to each other. In some embodiments, the plurality of connecting members 150 may extend between longitudinally adjacent circumferential bands of the plurality of circumferential bands 110. In some embodiments, the plurality of connecting members 150 may fixedly attach longitudinally adjacent circumferential bands of the plurality of circumferential bands 110 to each other. In some embodiments, the plurality of connecting members 150 may be oriented non-parallel to each other and/or the central longitudinal axis 102 of the endoprosthesis 100 in the radially collapsed configuration and in the radially expanded configuration. Other configurations are also contemplated.

In some embodiments, the plurality of connecting members 150 may be greater in density proximate the proximal end 104 of the endoprosthesis 100 than the distal end 106 of the endoprosthesis 100. In some embodiments, a density of the plurality of connecting members 150 may decrease in a distal direction and/or from the proximal end 104 of the endoprosthesis 100 toward the distal end 106 of the endoprosthesis 100. In some embodiments, ancillary procedures and/or devices may interact with the endoprosthesis 100 after implantation. The likelihood of interaction is greater at the proximal end 104 of the endoprosthesis 100. As such, it may be beneficial to have a greater density of the plurality of connecting members 150 (e.g., more connecting members) toward the proximal end 104 of the endoprosthesis 100 than the distal end 106 of the endoprosthesis 100 to provide adequate support between adjacent circumferential bands of the plurality of circumferential bands 110 and/or to prevent longitudinal deformation of the endoprosthesis 100.

In at least some embodiments, the endoprosthesis 100 may be balloon expandable. In one preferred but non-limiting example, the endoprosthesis 100 is balloon expandable. In some embodiments, the endoprosthesis 100 may be self-expanding. Other configurations, including combinations thereof, are also contemplated. The balloon may be fluidly couplable to a source of inflation fluid. In some embodiments, the balloon may be formed from a compliant material. In some alternative configurations, the balloon may be formed from a semi-compliant material or a non-compliant material. In some embodiments, the balloon may preferably be formed from a polymeric material. Some suitable but non-limiting examples of polymeric materials that may be used to form the balloon are discussed below. Other configurations and/or materials are also contemplated. Some additional suitable but non-limiting examples of materials that may be used to form the balloon, including metallic materials, composite materials, and the like, are also discussed below.

In some embodiments, the endoprosthesis 100 (e.g., the plurality of circumferential bands 110, the plurality of struts 120, the plurality of connecting members 150, etc.) may be formed and/or cut (e.g., via laser cutting, waterjet, machining, etc.) from a metallic tubular member. In some embodiments, the endoprosthesis 100 may be formed and/or cut from a flat sheet of material and rolled into a tubular shape and/or an annular shape, where the endoprosthesis 100 may be permanently fixed into the tubular shape and/or the annular shape, such as by welding, bonding, etc. Other configurations are also contemplated.

In some embodiments, the endoprosthesis 100 may preferably be formed from a metallic material, such as stainless steel, nickel-titanium alloy(s), etc. In one preferred but non-limiting example, the endoprosthesis 100 may be formed from a platinum-chromium (PtCr) alloy. Some additional suitable but non-limiting examples of metallic materials that may be used to form the endoprosthesis 100 are discussed below. Other configurations and/or materials are also contemplated. In some alternative embodiments, the endoprosthesis 100 may be formed from polymeric materials, composite materials, and the like, some non-limiting examples of which are discussed below.

In some embodiments, the endoprosthesis 100 may include a coating disposed thereon. In some embodiments, the endoprosthesis 100 may include a lubricious coating disposed thereon. In some embodiments, the endoprosthesis 100 may include a hydrophobic coating or a hydrophilic coating disposed thereon, depending on intended use and/or situational needs. In some embodiments, the endoprosthesis 100 may be coated with a medicament or drug disposed thereon and/or may include a drug-eluting coating disposed thereon. In one non-limiting example, the endoprosthesis 100 may comprise a polymeric coating configured to release and/or disperse one or more drugs, medicines, etc. Other configurations are also contemplated. Some suitable but non-limiting examples of coating types and/or materials that may be used with the endoprosthesis 100 are discussed below.

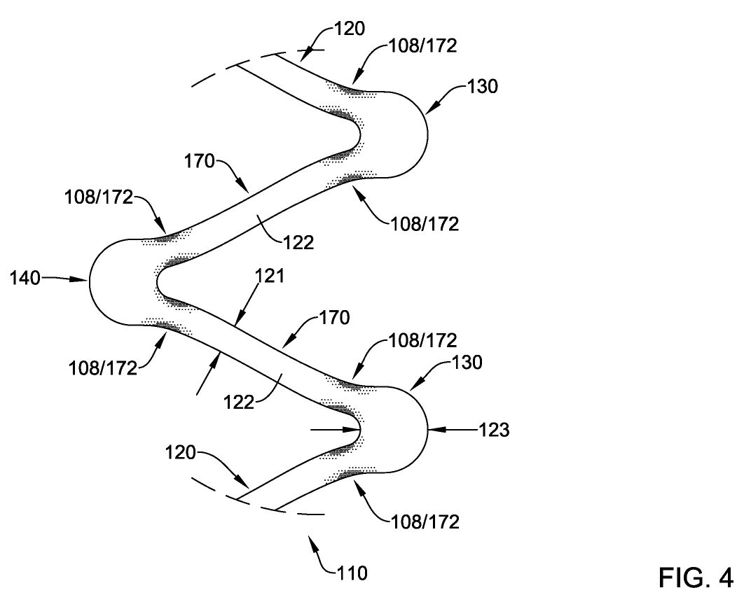

FIGS. 2-3 schematically illustrate the endoprosthesis 100 expanding from the radially collapsed configuration (e.g., FIG. 2) to the radially expanded configuration (e.g., FIG. 3). FIGS. 2-3 also illustrate stress points 108 and/or locations of concentrated stress or relatively high stress experienced by the endoprosthesis 100, the plurality of circumferential bands 110, the plurality of struts 120, the plurality of connecting members 150, etc. FIG. 4 illustrates a portion of the endoprosthesis of FIG. 3 in more detail. The stress points 108 and/or locations of concentrated stress or relatively high stress are shown by way of dotted shading, wherein in FIG. 4 a higher dot density indicates a higher amount of stress within the stress points 108. In some embodiments, the endoprosthesis 100 may be designed in a way that isolates stress to selected areas of the endoprosthesis 100, the plurality of circumferential bands 110, the plurality of struts 120, the plurality of connecting members 150, etc., which may be beneficial for reducing recoil (e.g., spring back) of the endoprosthesis 100 when an expanding force is removed (e.g., when the balloon expanding the endoprosthesis 100 is deflated).

In some embodiments, the plurality of struts 120 may have a first width 121 along a straight portion 122 of the plurality of struts 120, and the peaks 130 and/or the valleys 140 may have a second width 123 greater than the first width 121, as seen in FIG. 4. In some embodiments, the second width 123 may be 20-50% greater than the first width 121. These variations in second width 123 versus first width 121 (wherein the peaks 130 and/or the valleys 140 are 20-50% wider than the plurality of struts 120 and/or the straight portion 122) allow for localizing the expansion stress in targeted small structural segments, causing focal plastic deformations on them without stressing the rest of the architecture. Other variations and/or ranges of widths are also contemplated. In at least some embodiments, during expansion of the endoprosthesis 100, bending and/or deflection of the plurality of struts 120 and/or within the plurality of circumferential bands 110 may be “focused” to areas immediately adjacent the peaks 130 and the valleys 140 instead of bending and/or deflecting within the peaks 130 and/or the valleys 140 themselves. Bending and/or deflecting a smaller area may result in less recoil.

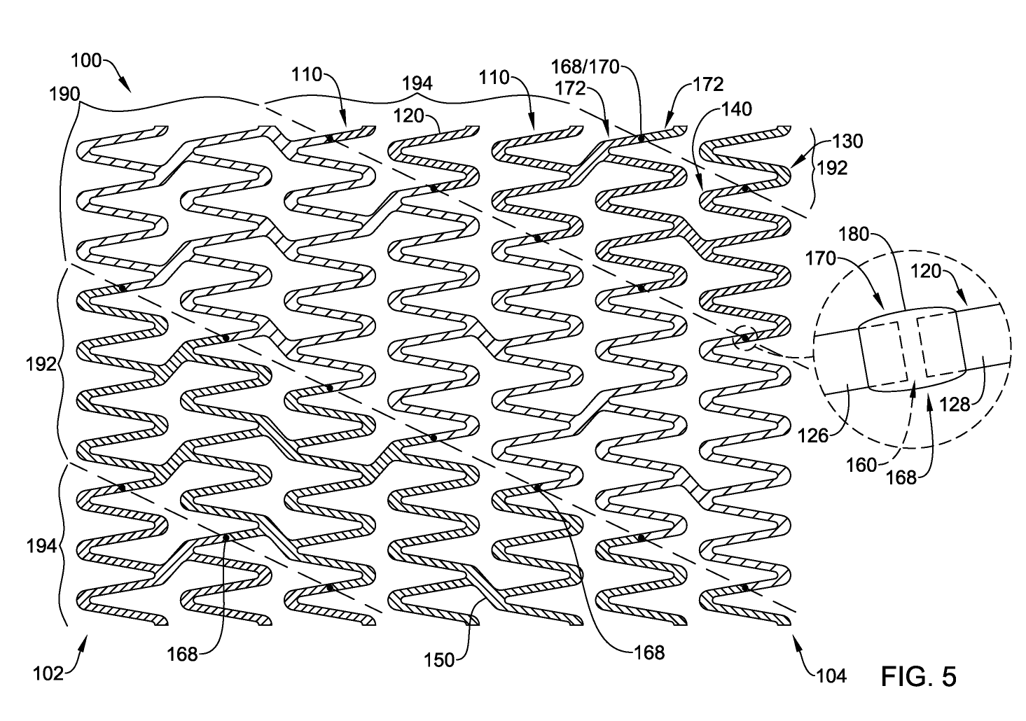

In order to facilitate restoration of vasomotion capabilities and/or function within the blood vessel or the body lumen being treated, the endoprosthesis 100 may be adapted and/or configured to separate from the substantially cylindrical structure into at least two, at least three, etc. distinct helical stent structures (e.g., FIG. 5) which may be configured to permit normal motion and/or dynamic function of the blood vessel or the body lumen after a predetermined period of time. However, locating individual points of separation 168 (e.g., FIG. 5) within the endoprosthesis 100 requires careful consideration of the desired function of the endoprosthesis 100 prior to separation and the mechanical properties (e.g., stress, strain, etc.) experienced by the endoprosthesis 100. For example, if too much stress is experienced at the individual points of separation 168 (e.g., FIG. 5), the stress may overcome the ability of the endoprosthesis 100 to remain coupled and the endoprosthesis 100 may separate prematurely, thereby reducing or eliminating the initial lumen-opening capability and/or function of the endoprosthesis 100 (e.g., the radially outward force exerted by the endoprosthesis 100 against the wall of the blood vessel or body lumen).

In some embodiments, each strut of a subset of the plurality of struts 120 may comprise a gap 160 formed at a location 170 experiencing or having relatively low stress during expansion of the endoprosthesis 100, as seen in FIG. 5. In some embodiments, locations 172 adjacent the location 170 experiencing or having relatively low stress during expansion of the endoprosthesis 100 may experience or have concentrated stress or relatively high stress during expansion of the endoprosthesis 100. In some embodiments, the location 170 experiencing or having relatively low stress during expansion of the endoprosthesis 100 may experience or have 50%-80% lower stress than the locations 172 experiencing or having concentrated stress or relatively high stress during expansion of the endoprosthesis 100. In some embodiments, the location 170 experiencing or having relatively low stress during expansion of the endoprosthesis 100 may experience or have 80%-100% lower stress than the locations 172 experiencing or having concentrated stress or relatively high stress during expansion of the endoprosthesis 100. Other configurations are also contemplated.

In some embodiments, the gap 160 may extend across an entire width (e.g., completely across the first width 121) of its respective strut of the plurality of struts 120. As discussed herein, each strut of the plurality of struts 120 may be defined by a peak (e.g., ref. 130) at one end and a valley (e.g., ref. 140) at an opposing end. In some embodiments, the location 170 experiencing or having relatively low stress during expansion of the endoprosthesis 100 may be disposed between the peak (e.g., ref. 130) and the valley (e.g., ref. 140) defining its respective strut of the plurality of struts 120. In some embodiments, the location 170 experiencing or having relatively low stress during expansion of the endoprosthesis 100 may be disposed equidistantly between the peak (e.g., ref. 130) and the valley (e.g., ref. 140) defining its respective strut of the plurality of struts 120.

In some embodiments, the locations 172 adjacent the location 170 experiencing or having relatively low stress, and experiencing or having concentrated stress or relatively high stress, during expansion of the endoprosthesis 100 may be disposed between the peak (e.g., ref. 130) and the valley (e.g., ref. 140) defining their respective strut of the plurality of struts 120. In some embodiments, the locations 172 adjacent the location 170 experiencing or having relatively low stress, and experiencing or having concentrated stress or relatively high stress, during expansion of the endoprosthesis 100 may be disposed immediately adjacent the peak (e.g., ref. 130) and the valley (e.g., ref. 140) defining their respective strut of the plurality of struts 120.

In some embodiments, each strut of the subset of the plurality of struts 120 may comprise a gap 160 formed along a straight portion 122 (e.g., FIG. 4) of the strut (e.g., ref. 120). The gap 160 may separate the strut (e.g., ref. 120) into a first portion 126 and a second portion 128 (e.g., FIG. 5). In some embodiments, the subset of the plurality of struts 120 may be arranged helically around the central longitudinal axis 102 of the endoprosthesis 100. In some embodiments, the subset of the plurality of struts 120 may divide the endoprosthesis 100 into at least two distinct helical stent structures. In some embodiments, the subset of the plurality of struts 120 may divide the endoprosthesis 100 into at least three distinct helical stent structures. Other configurations are also contemplated.

In some embodiments, the gap 160 may be formed and/or disposed at the location 170 experiencing or having relatively low stress during expansion of the endoprosthesis 100. In some embodiments, the locations 172 adjacent the location 170 experiencing or having relatively low stress, and experiencing or having concentrated stress or relatively high stress, during expansion of the endoprosthesis 100 may be disposed on opposing sides of the gap 160.

In at least some embodiments, the gap 160 may be filled with a biodegradable polymeric material 180. In some embodiments, the biodegradable polymeric material 180 may be configured to couple the first portion 126 of the strut (e.g., ref. 120) to the second portion 128 of the strut (e.g., ref. 120). In some embodiments, the biodegradable polymeric material 180 may be configured to fixedly secure the first portion 126 of the strut (e.g., ref. 120) to the second portion 128 of the strut (e.g., ref. 120). In some embodiments, the biodegradable polymeric material 180 may be configured to maintain the structural integrity of the endoprosthesis 100 during implantation and/or expansion from the radially collapsed configuration to the radially expanded configuration. In some embodiments, the biodegradable polymeric material 180 may be configured to maintain the structural integrity of the endoprosthesis 100 and/or the radially outward force exerted by the endoprosthesis 100 against the wall of the blood vessel or body lumen for a predetermined period of time post-implantation. During and/or after the predetermined period of time, the biodegradable polymeric material 180 may degrade in vivo. In some embodiments, the predetermined period of time may be several weeks, several months, etc. In one non-limiting example, the predetermined period of time may be about three months to about six months. Other configurations and/or periods of time are also contemplated. In some embodiments, the biodegradable polymeric material 180 may degrade gradually over the predetermined period of time. In some embodiments, the biodegradable polymeric material 180 may be configured to degrade rapidly after the predetermined period of time has passed. Other configurations are also contemplated.

In one preferred but non-limiting example, the biodegradable polymeric material 180 may be poly-l-lactic acid (PLLA). In another preferred but non-limiting example, the biodegradable polymeric material 180 may be poly lactic-co-glycolic acid (PLGA). Some suitable but non-limiting examples of biodegradable polymeric materials that may be used to fill the gap 160 are discussed below. Other configurations and/or materials are also contemplated.

As discussed herein, the endoprosthesis 100 may define at least two distinct helical stent structures, at least three distinct helical stent structures, etc. coupled together to form the substantially cylindrical structure. For the purpose of illustration only, FIG. 5 shows the endoprosthesis 100 (in the flat pattern view), having three distinct helical stent structures, wherein the three distinct helical stent structures are identified as a first distinct helical stent structure 190, a second distinct helical stent structure 192, and a third distinct helical stent structure 194 delineated via different hatching and separated by dashed lines. These designations are not intended to be limiting. After the predetermined period of time and/or after degradation of the biodegradable polymeric material 180, the substantially cylindrical structure of the endoprosthesis 100 may be configured to separate at the individual points of separation 168 and/or the gap(s) 160 such that the three distinct helical stent structures (e.g., the first distinct helical stent structure 190, the second distinct helical stent structure 192, and the third distinct helical stent structure 194) separate from each other into independent structures to enable, facilitate, and/or promote the restoration of vasomotion capabilities and/or function within the blood vessel or the body lumen being treated.

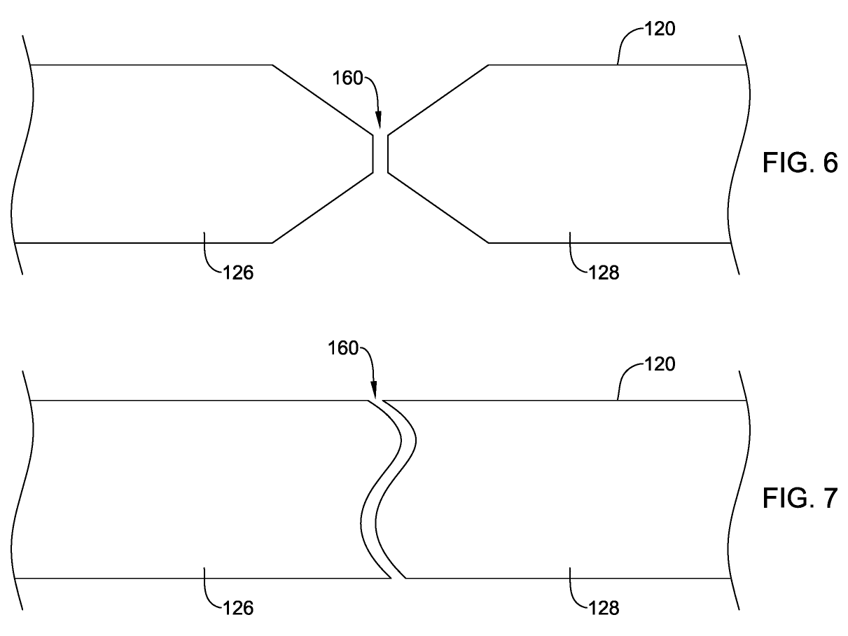

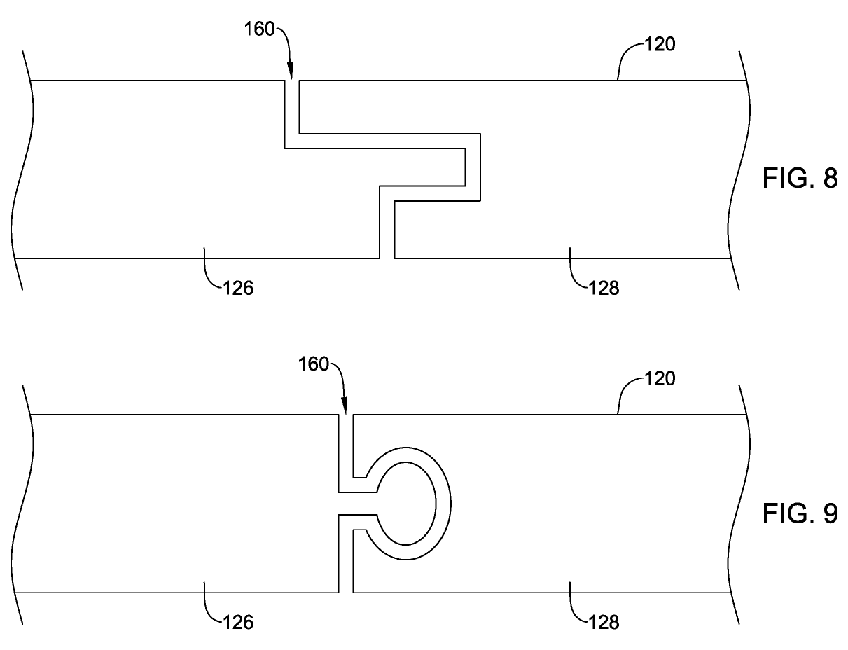

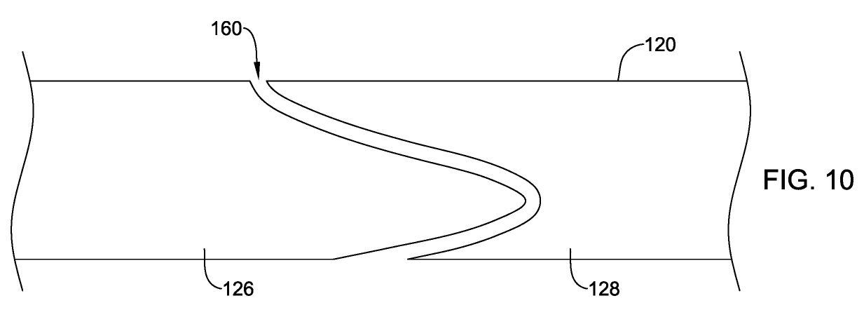

In some embodiments, the gap 160 may be formed as a straight cut between the first portion 126 and the second portion 128 of the strut (e.g., ref. 120), as in the example of FIG. 5. Other examples and/or alternatives are also contemplated, and in some embodiments, may depend on the precision of the cutting tool used to form the gap 160. FIG. 6 illustrates the gap 160 as tapered cuts on the first portion 126 and the second portion 128 of the strut (e.g., ref. 120). FIG. 7 illustrates the gap 160 as a regular curved (e.g., sinusoidal) cut between the first portion 126 and the second portion 128 of the strut (e.g., ref. 120). FIG. 8 illustrates the gap 160 as an irregular “notch” cut between the first portion 126 and the second portion 128 of the strut (e.g., ref. 120). FIG. 9 illustrates the gap 160 as a complex interlocking cut between the first portion 126 and the second portion 128 of the strut (e.g., ref. 120). In some embodiments, the complex interlocking cut may add and/or enhance the strength of the coupling between the first portion 126 and the second portion 128 of the strut (e.g., ref. 120), if so desired for a particular application. FIG. 10 illustrates the gap 160 as an irregular curved cut between the first portion 126 and the second portion 128 of the strut (e.g., ref. 120). Other configurations are also contemplated. All of the cut patterns (visualized in FIGS. 6-10) for the gap 160 may enhance in different degrees the strength of the coupling between the first portion 126 and the second portion 128 of the strut (e.g., ref. 120) and allow for complete structural integrity of the architecture of the endoprosthesis 100 when the gap 160 is filled with the biodegradable polymeric material 180. Some configurations may be relatively easier to decouple and/or unlock than others.

The level of stress experienced during expansion of the endoprosthesis 100 may be proportional to the opening of the angulation in the zig-zag pattern of the scaffolding architecture, wherein a greater angle correlates to greater stress. The architecture of the endoprosthesis 100 features a peak (e.g., ref. 130) and/or a valley (e.g., ref. 140) wider than the strut (e.g., ref. 120), which re-directs the expansion stress to cause plastic deformation in two symmetrical small architectural segments on opposing sides of the peak (e.g., ref. 130) and/or the valley (e.g., ref. 140), leaving relatively unstressed the middle of the strut (e.g., the straight portion 122 of the plurality of struts 120) relatively unstressed independently of the achieved angle of the zig-zag pattern. This is where the gap 160 is formed (e.g., within the straight portion 122) and the biodegradable polymeric material 180 is applied to fill the gap 160. The Young’s Modulus for each material may differ (e.g., metallic struts versus polymeric material) and therefore, the design avoids high stress in the gap 160 where biodegradable polymeric material 180 joins the first portion 126 and the second portion 128 of the plurality of struts 120. In some embodiments, the proximal end 104 of the endoprosthesis 100 may require further post-dilation and overexpansion if the target site and/or body lumen is tapered, so the proximal end 104 of the endoprosthesis 100 may be subjected to higher stress than the distal end 106 and at and/or adjacent the proximal end 104 the zig-zag pattern may be opened to a greater angulation.

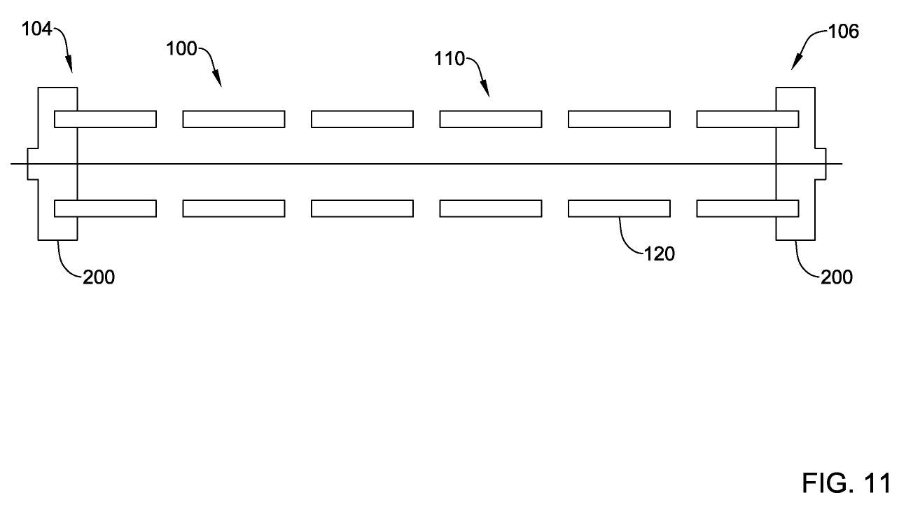

FIGS. 11-16 illustrate selected aspects of a method or methods of making the endoprosthesis 100 in a highly schematic manner to facilitate ease of understanding. In some embodiments, the method of making the endoprosthesis 100 may comprise forming the plurality of circumferential bands 110 and the plurality of connecting members 150, as described herein and shown in more detail in other figures, from a metallic tubular member to form the endoprosthesis 100. In some embodiments, the endoprosthesis 100 may be formed in the radially collapsed configuration. In at least some embodiments, the endoprosthesis 100 may be cut from the metallic tubular member, such as via laser cutting, machining, etc. In some alternative embodiments, the endoprosthesis 100 may be formed from a piece of flat stock and then rolled to form a tubular shape. A portion of the endoprosthesis 100 is shown in the figures with a simplified longitudinal profile, in the radially collapsed configuration.

In some embodiments, the endoprosthesis 100 may be clamped at the proximal end 104 and at the distal end 106, as seen in FIG. 11. The clamps 200 may be configured to facilitate rotation and alignment of the endoprosthesis 100. In some embodiments, the clamps 200 may be configured to provide high precision and accuracy for subsequent operations and/or steps.

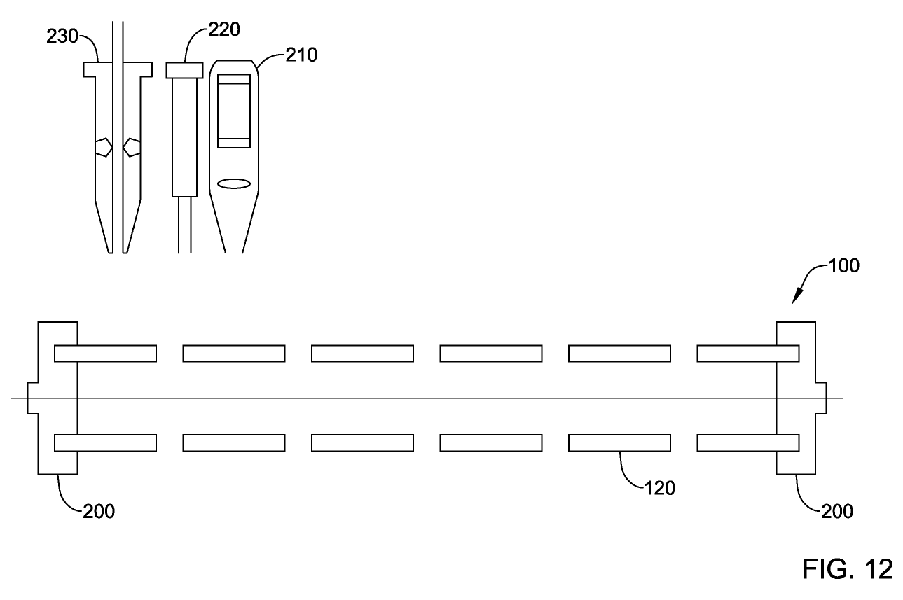

In some embodiments, the method of making the endoprosthesis 100 may use a plurality of tools and/or pieces of equipment, as seen in FIG. 12. For the purpose of illustration only, the plurality of tools and/or pieces of equipment are shown schematically, and the representations used may not be reflective of actual tools and/or pieces of equipment. In some embodiments, the method of making the endoprosthesis 100 may use a laser cutter 210. In some embodiments, the method of making the endoprosthesis 100 may use a plasma tool 220. In some embodiments, the method of making the endoprosthesis 100 may use a polymeric material applicator 230. In some embodiments, the polymeric material applicator 230 may include an ink-jet injector. In some embodiments, the plurality of tools and/or pieces of equipment (e.g., the laser cutter 210, the plasma tool 220, the polymeric material applicator 230, etc.) may be positioned and/or arranged in a group such that the plurality of tools and/or pieces of equipment (e.g., the laser cutter 210, the plasma tool 220, the polymeric material applicator 230, etc.) move together relative to the endoprosthesis 100. In some embodiments, the plurality of tools and/or pieces of equipment (e.g., the laser cutter 210, the plasma tool 220, the polymeric material applicator 230, etc.) may be positioned and/or arranged independently of each other such that the plurality of tools and/or pieces of equipment (e.g., the laser cutter 210, the plasma tool 220, the polymeric material applicator 230, etc.) move independently (e.g., one at a time) relative to the endoprosthesis 100. Other configurations, including combinations thereof, are also contemplated. It will be understood that relative movement may be provided by moving the endoprosthesis 100 and/or the clamps 200 relative to the plurality of tools and/or pieces of equipment (e.g., the laser cutter 210, the plasma tool 220, the polymeric material applicator 230, etc.).

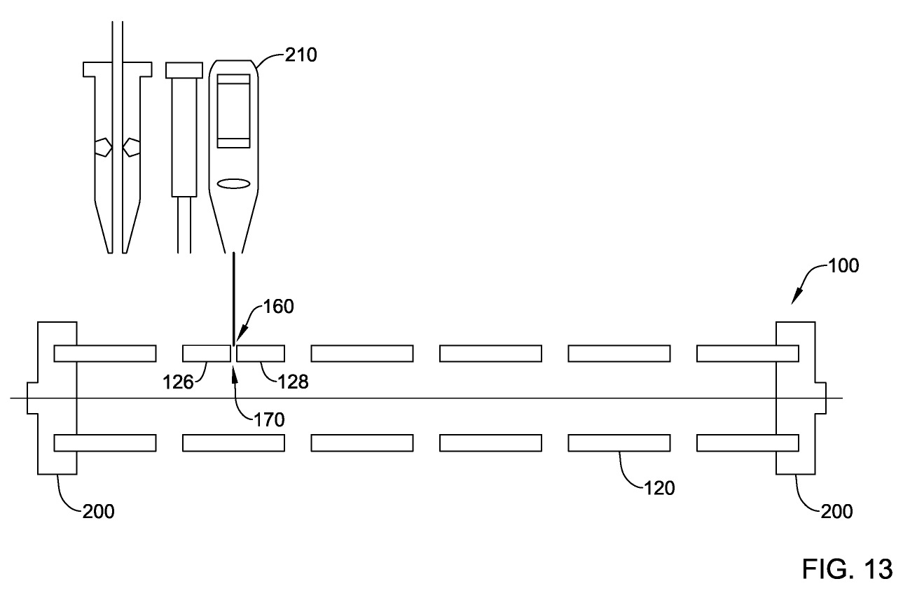

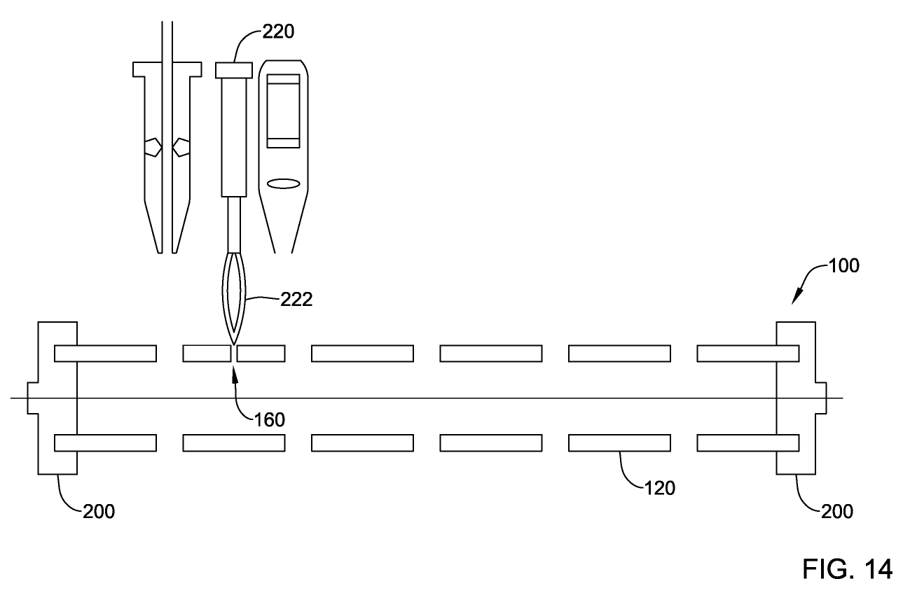

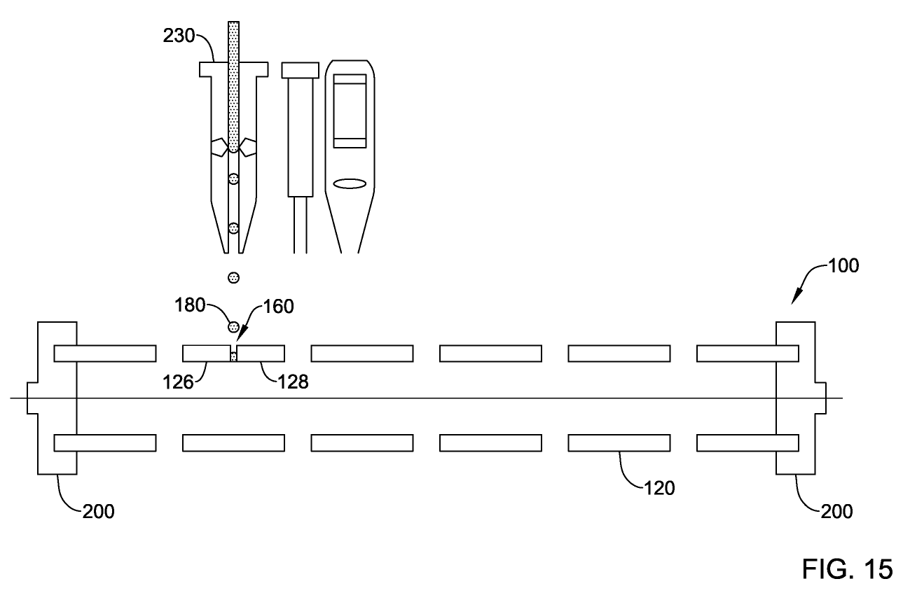

In some embodiments, the method of making the endoprosthesis 100 may comprise cutting a first strut (e.g., ref. 120) of the plurality of struts 120 to form a first gap (e.g., ref. 160) at a location 170 along the first strut (e.g., ref. 120) of the plurality of struts 120 experiencing or having relatively low stress during expansion of the endoprosthesis 100, as seen in FIG. 13. As discussed herein, the first gap (e.g., ref. 160) may separate the first strut (e.g., ref. 120) of the plurality of struts 120 into a first portion 126 and a second portion 128. In some embodiments, the method of making the endoprosthesis 100 may comprise applying a plasma plume 222 to the first gap (e.g., ref. 160) using the plasma tool 220 to clean the first gap (e.g., ref. 160), as seen in FIG. 14. In some embodiments, after cleaning the first gap (e.g., ref. 160) with the plasma plume 222, the method of making the endoprosthesis 100 may comprise filling the first gap (e.g., ref. 160) with a biodegradable polymeric material 180 to couple the first portion 126 of the first strut (ref. 120) to the second portion 128 of the first strut (ref. 120), as seen in FIG. 15.

In some embodiments, after filling the first gap (e.g., ref. 160), the method of making the endoprosthesis 100 may comprise cutting a second strut (e.g., ref. 120) of the plurality of struts 120 to form a second gap (e.g., ref. 160) at a location (e.g., ref. 170) along the second strut (e.g., ref. 120) of the plurality of struts 120 experiencing or having relatively low stress during expansion of the endoprosthesis 100. As discussed herein, the second gap (e.g., ref. 160) may separate the second strut (e.g., ref. 120) of the plurality of struts 120 into a first portion (e.g., ref. 126) and a second portion (e.g., ref. 128). In some embodiments, the method of making the endoprosthesis 100 may comprise applying a plasma plume 222 to the second gap (e.g., ref. 160) using the plasma tool 220 to clean the second gap (e.g., ref. 160). In some embodiments, after cleaning the second gap (e.g., ref. 160) with the plasma plume 222, the method of making the endoprosthesis 100 may comprise filling the second gap (e.g., ref. 160) with the biodegradable polymeric material 180 to couple the first portion (e.g., ref. 126) of the second strut (e.g., ref. 120) to the second portion (e.g., ref. 128) of the second strut (e.g., ref. 120). This process may be repeated as necessary and/or desired to produce the desired arrangement of the endoprosthesis 100. In order to maintain the structural integrity of the endoprosthesis 100, it may be important to process the endoprosthesis 100 in a way that cuts and fills each gap (e.g., ref. 160) individually and/or one at a time before proceeding to the next gap (e.g., ref. 160). In some alternative embodiments, the second strut (e.g., ref. 120) of the plurality of struts 120 and/or the second gap (e.g., ref. 160) may be cut while (e.g., simultaneously with) the first gap (e.g., ref. 160) is being filled with the biodegradable polymeric material 180.

In some embodiments, the method of making the endoprosthesis 100 may comprise cutting a subset of struts of the plurality of struts 120 to form gaps (e.g., ref. 160) at locations (e.g., ref. 170) experiencing or having relatively low stress during expansion of the endoprosthesis 100. In at least some embodiments, the subset of struts of the plurality of struts 120 may be arranged helically around the central longitudinal axis 102 of the endoprosthesis 100. In some embodiments, the method of making the endoprosthesis 100 may comprise filling the gaps (e.g., ref. 160) with the biodegradable polymeric material 180, wherein each gap (e.g., ref. 160) is cut and then filled with the biodegradable polymeric material 180 prior to cutting a subsequent gap (e.g., ref. 160).

In some embodiments, the endoprosthesis 100 may define at least three distinct helical stent structures (e.g., FIG. 5; refs. 190, 192, 194) coupled together by the biodegradable polymeric material 180 to form the substantially cylindrical structure. In some embodiments, the biodegradable polymeric material 180 may be configured to degrade after a predetermined period of time, thereby permitting the at least three distinct helical stent structures (e.g., FIG. 5; refs. 190, 192, 194) to separate from each other. In at least some embodiments, the biodegradable polymeric material 180 may be configured to degrade only in vivo. Other configurations are also contemplated.

In some embodiments, the cutting and filling steps of the method of making the endoprosthesis 100 may occur while the endoprosthesis 100 is disposed in the radially collapsed configuration. In some embodiments, the plurality of circumferential bands 110 and the plurality of connecting members 150 may be monolithically formed with each other prior to cutting any struts (e.g., ref. 120) of the plurality of struts 120 and/or prior to forming any gaps (e.g., ref. 160).

In some embodiments, all helical stent structures of the endoprosthesis 100 (e.g., all of the at least two helical stent structures, all of the at least three helical stent structures, etc.) may remain interconnected throughout the method of making the endoprosthesis 100. For example, all helical stent structures of the endoprosthesis 100 (e.g., all of the at least two helical stent structures, all of the at least three helical stent structures, etc.) may remain interconnected during all cutting and filling steps of the method of making the endoprosthesis 100 to prevent the endoprosthesis 100 and/or the substantially cylindrical structure from falling apart. Therefore, the structural integrity of the endoprosthesis 100 and/or the substantially cylindrical structure is maintained during the method of making the endoprosthesis 100.

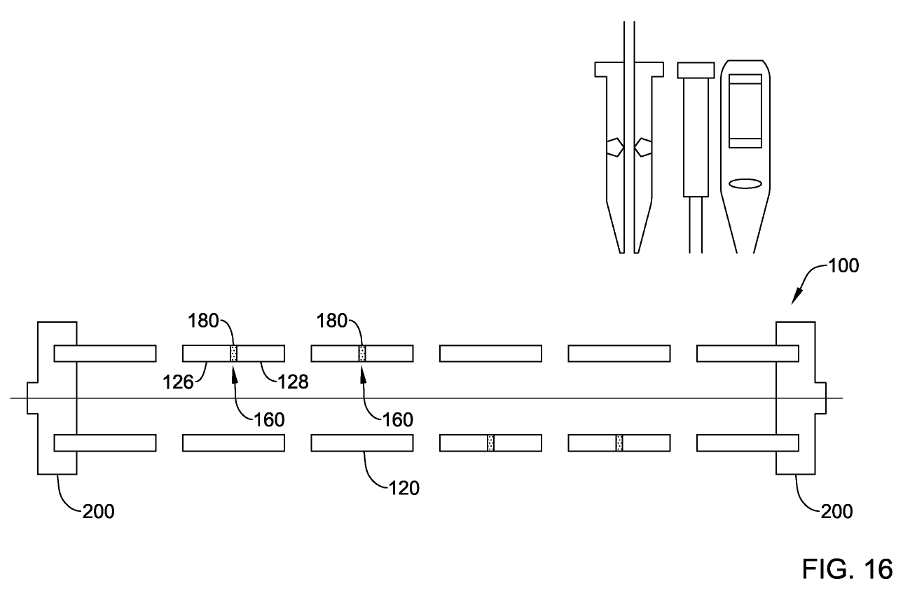

FIG. 16 illustrates a portion of the endoprosthesis 100 upon completion of the cutting and filling steps of the method of making the endoprosthesis 100. As may be seen in FIG. 16, the endoprosthesis 100 may comprise a plurality of gaps (e.g., ref. 160) that have been filled with the biodegradable polymeric material 180 in a generally helical pattern and/or arrangement. Finally, after all cutting and filling steps have been completed, the endoprosthesis 100 may be released from the clamps 200. In some embodiments, additional processing and/or other finishing steps may be performed after the endoprosthesis 100 has been released from the clamps 200. For example, in some embodiments, a coating step may be performed after the stent has been released from the clamps 200. Other configurations are also contemplated.

The materials that can be used for the various components of the endoprosthesis and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the endoprosthesis, the plurality of circumferential bands, the plurality of struts, the plurality of connecting members, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, bioabsorbable polymers (for example, poly-l-lactic acid (PLLA), poly lactic-co-glycolic acid (PLGA), etc.), other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis configured to shift between a radially collapsed configuration and a radially expanded configuration, comprising:
a plurality of circumferential bands, wherein each circumferential band comprises a plurality of struts joined in a serpentine pattern to define peaks and valleys; and
a plurality of connecting members extending between adjacent circumferential bands of the plurality of circumferential bands;
wherein each strut of a subset of the plurality of struts comprises a gap formed at a location having low stress during expansion of the endoprosthesis;
wherein locations adjacent the location having low stress experience high stress during expansion of the endoprosthesis.

2. The endoprosthesis of claim 1, wherein the gap extends across an entire width of its respective strut.

3. The endoprosthesis of claim 1, wherein each strut is defined by a peak at one end and a valley at an opposing end.

4. The endoprosthesis of claim 3, wherein the location having low stress during expansion of the endoprosthesis is disposed between the peak and the valley.

5. The endoprosthesis of claim 4, wherein the location having low stress during expansion of the endoprosthesis is disposed equidistantly between the peak and the valley.

6. The endoprosthesis of claim 3, wherein the locations adjacent the location having low stress during expansion of the endoprosthesis are disposed between the peak and the valley.

7. The endoprosthesis of claim 6, wherein the locations adjacent the location having low stress during expansion of the endoprosthesis are disposed immediately adjacent the peak and the valley.

8. The endoprosthesis of claim 1, wherein the plurality of struts has a first width, and the peaks and valleys have a second width greater than the first width.

9. The endoprosthesis of claim 1, wherein the gap is filled with a biodegradable polymeric material.

10. The endoprosthesis of claim 1, wherein the plurality of circumferential bands and the plurality of connecting members are cut from a metallic tubular member.

11. The endoprosthesis of claim 1, wherein the subset of the plurality of struts is arranged helically around a central longitudinal axis of the endoprosthesis.

12. The endoprosthesis of claim 1, wherein the subset of the plurality of struts divides the endoprosthesis into at least two distinct helical stent structures.

13. A method of making an endoprosthesis configured to shift between a radially collapsed configuration and a radially expanded configuration, comprising:
forming a plurality of circumferential bands, wherein each circumferential band comprises a plurality of struts joined in a serpentine pattern to define peaks and valleys, and a plurality of connecting members extending between adjacent circumferential bands of the plurality of circumferential bands from a metallic tubular member to form the endoprosthesis;
cutting a first strut of the plurality of struts to form a first gap at a location along the first strut having low stress during expansion of the endoprosthesis;
filling the first gap with a biodegradable polymeric material;
after filling the first gap, cutting a second strut of the plurality of struts to form a second gap at a location along the second strut having low stress during expansion of the endoprosthesis; and
filling the second gap with the biodegradable polymeric material.

14. The method of claim 13, further comprising:
cutting a subset of struts of the plurality of struts to form gaps at locations having low stress during expansion of the endoprosthesis, wherein the subset of struts is arranged helically around a central longitudinal axis of the endoprosthesis; and
filling the gaps with the biodegradable polymeric material;
wherein each gap is cut and then filled prior to cutting a subsequent gap.

15. The method of claim 13, wherein the endoprosthesis defines at least three distinct helical stent structures coupled together by the biodegradable polymer to form a substantially cylindrical structure.

16. The method of claim 15, wherein the biodegradable polymeric material is configured to degrade after a predetermined period of time, thereby permitting the at least three distinct helical stent structures to separate from each other.

17. The method of claim 13, wherein the cutting and filling steps occur while the endoprosthesis is disposed in the radially collapsed configuration.

18. The endoprosthesis of claim 13, wherein the plurality of circumferential bands and the plurality of connecting members are monolithically formed with each other prior to cutting any struts.

19. An endoprosthesis configured to shift between a radially collapsed configuration and a radially expanded configuration, comprising:
a plurality of circumferential bands, wherein each circumferential band comprises a plurality of struts joined in a serpentine pattern to define peaks and valleys; and
a plurality of connecting members extending between adjacent circumferential bands of the plurality of circumferential bands;
wherein each strut of a subset of the plurality of struts comprises a gap formed along a straight portion of the strut, the gap separating the strut into a first portion and a second portion;
wherein the gap is filled with a biodegradable polymeric material coupling the first portion to the second portion.

20. The endoprosthesis of claim 19, wherein the plurality of circumferential bands and the plurality of connecting members are formed from a metallic tubular member.
